(19) United States
(12) Patent Application Publication
(10) Pub. No.: US 2002/0146463 A1
(43) Pub. Date: Oct. 10, 2002

(54) Health promoting compositions

(76) Inventor: Paul Clayton, London (GB)

Correspondence Address: Finnegan, Henderson, Farabow,, Garrett & Dunner, L.L.P., 1300 I Street, N.W., Washington, DC, 20005-3315, US

(21) Appl. No.: 10/014,488

(22) Filed: Dec. 14, 2001

(30) Foreign Application Priority Data
Mar. 1, 2001 (DE) 101 09 798.0
Dec. 16, 2000 (EP) 00127644.3

(51) Int. Cl. A61K 38/28; A61K 31/015; A61K 31/355; A61K 33/34; A61K 33/32

(52) U.S. Cl. 424/617; 424/630; 424/643; 424/655; 514/3; 514/54; 514/393; 514/23; 514/458; 514/474; 514/725; 514/682; 514/763

(57) ABSTRACT

The present invention discloses compositions useful as food additives or in medicaments to improve health by treating, preventing, or curing diseases and medical disorders. In particular, the compositions are formulated to provide the nutrients necessary for maintaining optimal free radical defenses in the body.

### FIELD OF THE INVENTION

The present invention relates to compositions that are useful as food additives or in medicaments to improve health by treating, preventing, or curing diseases and medical disorders.

### BACKGROUND

An explosion is an extremely fast fire. Human metabolism is also a type of fire, albeit a very slow and controlled fire, which consumes oxygen and carbon compounds to produce water, carbon dioxide, ATP (a molecule of energy), and free radicals. The low heat produced in this slow fire keeps us warm and alive.

To stop the fire from burning out of control and damaging us, we tend it very carefully. For example, the fire of metabolism is separated into tiny, enzyme-regulated steps, surrounded with firebreaks, and protected by fire extinguishers in every corner to douse excess free radicals. The fire extinguishers, which protect against free radicals in the body, are anti-oxidants.

Free radicals can cause illness and premature death. Thus, boosting the body's defenses against free radicals may improve health and life span. For example, animal studies suggest that high doses of anti-oxidants may prolong life significantly.

There are at least three important components of a body's anti-oxidant defenses.

1) Trace Minerals: anti-oxidant enzymes are made in the body and contain an atom of selenium, zinc, manganese, copper or iron. Thus, small amounts of these metal minerals are vital to optimal health.

2) Vitamins and vitamin-like compounds: anti-oxidant micro-nutrients are obtained from our diet. These include vitamins A, C and E, and the B vitamins. Co-enzyme Q10, flavonoids and carotenoids are important vitamin-like compounds which also have anti-oxidant properties.

3) Anti-oxidants: anti-oxidant compounds are formed in the body, and are made up from elements in the diet. These include melatonin, glutathione, oestrogen, lipoic acid, Q10 and others.

Very few people eat a diet containing enough of the trace minerals, vitamins and other anti-oxidant compounds described above. Nevertheless, it's important for the body to get sufficient quantities of these substances, as they work best in combination to defend against free radicals. A dietary deficiency of these substances may be a primary reason why many people die of free radical-related disease, such as cancer.

There are anti-oxidants in every living tissue, in every organ and cell of our body, and in almost every food we eat. Meat, fish, poultry, milk, eggs, vegetables, fruit, nuts, grains, and legumes all contain anti-oxidants. If they didn't, these foods would rapidly oxidize and turn rancid before being incorporated into our diets.

Unfortunately, the major anti-oxidants in meat, milk and eggs are destroyed by cooking. Nor is there much point in eating raw eggs and steak tartare, because even if the anti-oxidants survive the cooking process, they would be broken down in the digestive tract.

Anti-oxidants in fruits and vegetables are more likely to survive the cooking process (although they are destroyed if cooked into a pulp; thus, lightly cooking them is best). In addition, anti-oxidants in fruits and vegetables are generally well absorbed by the body, although some people are able to absorb them better than others.

Clearly, dietary factors exert an enormous influence on the levels of free radicals in our bodies. But there are also many non-dietary lifestyle factors, such as smoking, sunbathing and aerobic exercise, which increase the amount of free radicals in our bodies.

Chronic infections, whether bacterial, viral, or fungal, can also cause increased free radical formation. Such infections are linked to increased DNA damage and increased risk of cancer. In this regard, some bacteria and viruses appear to be more dangerous than others. For example, Helicobacter pylori as a cause of stomach cancer and papilloma virus as a cause of cervical cancer are among the best documented.

The body's first response to increased amounts of free radicals, whatever their origin, is to defend itself by increasing levels of anti-oxidant enzymes. This same defensive response is shared by many different species in the plant and animal kingdoms.

To maintain optimal health, most dietary advice concentrates on the benefits of increasing the intake of anti-oxidant vitamins. However, this is not sufficient by itself. The major anti-oxidant enzymes produced by the body require other substances from the diet. For example, each enzyme typically requires an atom of zinc, iron, selenium, copper, or manganese to function properly; and these minerals must also be obtained from food. Unfortunately, evidence suggests that there is widespread depletion of one or more of these vital trace elements in a typical diet. Due to this depletion of micro-nutrients, many people have sub-optimal anti-radical defenses.

Impaired anti-oxidant defenses due to dietary deficiencies are a significant cause of ill health. Thus, governments may be better served by spending less money on treating illness and more money on prevention through better nutritional education. Together with improved food labelling, properly designed food fortification programs, and health-orientated agricultural policies (which shift subsidies from meat and dairy production to fruit and vegetable growers) the health of entire nations may be improved.

Unfortunately, waiting for governments to establish new policies or reform current policies can take a long time, especially when there is strong opposition to change from groups desiring to maintain the status quo. However, there is one simple nutritional step we can all take to improve our anti-oxidant defenses; one which would greatly increase our chances of living a long and healthy life: eat more fruit and vegetables.

But even this is no guarantee of achieving optimal nutrition. With the exception of Co-enzyme Q10, most vitamin micro-nutrients are predominantly derived from plant foods, which is why it's good idea to eat more fruit, vegetables, nuts, grains and legumes. However, the trace anti-oxidant metals, and some anti-oxidant compounds (e.g., the carotenoids), are not exclusive to plants; they can also be found in meat. Unfortunately, they tend to concentrate in parts of the animal that people do not normally eat. For example, liver and kidney (and testes and brain) are among the best sources of trace metals and some vitamins (e.g., A, K, E and Co-enzyme Q10). However, such meats are becoming more unpopular for human consumption; and in many affluent cultures they are predominantly used for pet food. The meat that is normally consumed by humans is mostly skeletal muscle, which is not as good of a source of many micro-nutrients.

Another source of dietary deficiency involves depleted soils, which produce crops low in specific minerals. This is the main cause of the selenium depletion, which occurs throughout much of Northern Europe, mainland China, parts of Africa, and elsewhere. People living in areas where the soil is depleted, eating foods imported from such areas, or eating a lot of processed foods are likely to have sub-optimal anti-oxidant enzyme defenses due to dietary deficiency. The combination of impaired anti-oxidant defenses and increased exposure to free radicals is a recipe for premature aging and illness.

Anti-oxidants work best to prevent disease when given together, rather than as monotherapies. Foods naturally provide complex mixtures of anti-oxidants; and before the arrival of the supplement industry, we obtained all our anti-oxidants from food. Supplements are not complete sources of the nutrients that naturally occur in foods. For example, many supplements contain beta carotene, but there are over 600 carotenoids in fruits and vegetables. Thus, choosing a single carotenoid does not mimic the naturally occuring forms. Although beta carotene is the main carotenoid in most diets, our bodies also contain, and probably need, alpha carotene (from carrots or pumpkins), lutein (green leaf vegetables, especially kale and broccoli), lycopene (tomatoes), cryptoxanthin (oranges), zeaxanthin (red pepper and spinach), and others. Thus, supplements may not be an adequate source of nutrients that are necessary to achieve optimal anti-oxidant defenses.

Many studies have linked deficiencies in particular micro-nutrients with certain diseases. For example, low selenium has been linked to arthritis and myocardial disease in China, coronary artery disease in Finland, breast cancer in New Zealand, and goiter in Zaire. However, these studies generally consider the effects of single anti-oxidants, rather than the effects of a combination of anti-oxidant compounds.

Despite these promising headlines, it is not very helpful to single out any one micro-nutrient (such as lycopene or selenium) as being “anti-cancer.” Rather, it is much more important to consider the overall nutritional status of an individual or group of people. And such assessments must take into account the typical nutritional profile for each country or region being assessed, because the patterns of illness caused by a particular deficiency may vary from one population to the next.

Clearly there exists a need in the art for compositions that provide the proper amounts of nutritional components necessary to maintain optimal health, and in particular, provide stronger antioxidant defenses.

### DESCRIPTION OF THE INVENTION

The present invention is directed to one or more compositions that may be used as food additives or medicaments to treat, reduce or inhibit particular diseases. Preferably, the composition provides nutrients that are used by the body to maintain optimal free radical defenses. The compositions of the present invention are particularly useful for incorporation of anti-oxidants into nutritional programs to treat or cure specific illnesses; and also for preventing disease in healthy people.

Scientists use a number of different tests to measure the anti-oxidant effectiveness of different compounds. From these tests, a few general patterns have emerged.

Vitamins and other compounds in the body are generally used up in a particular order. For example, many tests show that vitamin C tends to be used up first. When the vitamin C is gone, the carotenoid compounds are used next, then vitamin E; and when the E has all been used up, lipid oxidation begins. Adding either vitamins C or E helps prevent lipid oxidation, but does not protect proteins from oxidative damage. To protect proteins from oxidation, other anti-oxidants such as flavonoids or glutathione (discussed below) are needed. Due to this complexity, monotherapy (i.e., attempting to prevent free radical damage with a single anti-oxidant) is both theoretically and experimentally unsound.

In one embodiment, the presently disclosed compositions comprise beta carotene in an amount of about 10-15 mg/day. The beta carotene may be from any source, natural or synthetic, but is preferably a mixed, natural source of carotenoids. Beta carotene is also typically combined with Vitamin C. An individual taking this amount of beta carotene is in the top few percent of the population, a group which appears to be at reduced risk of oral and colon cancers, coronary artery disease, and cataracts. Larger doses are also generally safe. For example, 500 patients with skin conditions took 180-300 mg beta carotene/day for 10 years, without report of adverse effects. However, smokers and others at risk of lung cancer must be cautious with beta carotene.

In another embodiment, the present composition comprises beta carotene in combination with other carotenoids. Examples of suitable carotenoids, and the appropriate amounts for their use, include about 6 mg lycopene and about 6 mg lutein per day. Beta carotene is also preferably combined with vitamin C.

In another embodiment, the presently disclosed compositions may comprise vitamin E. The vitamin E may be provided at about 400 IU (265 mg)/day. The vitamin E may be derived from any source, natural or synthetic. In a preferred embodiment, vitamin E is derived from a natural source. The American Physicians Trial and other data suggest that 265 mg/day is an optimal dose of vitamin E for coronary disease. The preferred form of vitamin E is the natural form that contains only one isomer, D-alpha; whereas synthetic vitamin E contains eight isomers.

The natural form of vitamin E is preferred because it is believed that the D-alpha isomer inhibits the proliferation of smooth muscle cells in blood vessel walls (a highly desirable anti-coronary effect), while some of the other isomers in synthetic vitamin E block this property, and may therefore be cardiotoxic.

Very high doses of vitamin E are probably not useful and may be potentially harmful if used without other anti-oxidants. For example, it may inhibit the absorption of beta carotene and Vitamin K.

The presently disclosed compositions may be used to treat heart disease and related illnesses. For example, the current theory of coronary artery disease centers on the oxidization of LDL (low-density lipoprotein) cholesterol particles in the blood. The lipid and cholesterol products resulting from the oxidation of LDL cholesterol attack the artery walls, increasing the risk of clotting and raising blood pressure. This ultimately increases the risk of heart attacks.

Many anti-oxidants help protect LDL cholesterol from oxidation. For example, when supplements of selenium and vitamins C and E are given to human volunteers, their cholesterol becomes more resistant to oxidation. Moreover, vitamin E has been shown to reduce the risk of heart attacks by more than half in three recent trials. Studies also suggest that vitamin E reduces the progression of atheroma (furring of the arteries) in ways which are now well understood. Thus, vitamin E may be important to maintain proper health.

However, taking vitamin E by itself may not be adequate. In fact, under certain conditions, Vitamin E can accelerate the oxidation of LDL cholesterol unless there is enough Vitamin C (or flavonoids) around to protect the Vitamin E itself from being oxidized. This is probably one of the main cardioprotective roles of Vitamin C. Vitamin C also helps to maintain the lining of the arteries and reduces levels of clotting factors in the blood. It was previously believed that Vitamin C could increase levels of the ‘good’ HDL (high-density lipoprotein) cholesterol, but this now looks unlikely.

All this means that Vitamin E is preferably taken with Vitamin C, Co-enzyme Q10 (which appears to be a critical anti-oxidant in LDL), and the metal trace elements manganese, zinc and copper (which help the anti-oxidant enzymes to function properly), because all of these anti-oxidant defense systems work together. Thus, the presently disclosed compositions may contain one or more of these compounds, alone or in combination with each other. This sort of combination will undoubtedly help to prevent LDL oxidation in the plasma and slow the migration of oxidized cholesterol from the plasma into the artery walls.

In anyone over the age of 20, however, some oxidized LDL cholesterol is already inside the artery walls. Thus, we need different anti-oxidants to stop the disease process. Flavonoids serve this function. These anti-oxidants are able to get into the arterial walls, and slow or stop the progression of atheroma there. Beta carotene may be important, too. It may not protect circulating LDL from oxidation, but it prevents cells in the blood vessels' walls from further oxidizing the LDL that is already there.

The evidence that anti-oxidants reduce the risk of coronary artery disease is overwhelming. In fact, some eminent scientists now say that poor anti-oxidant status is a better predictor of the risk of heart attacks than high cholesterol levels, blood pressure, or any other of the known risk factors. Thus, the presently disclosed compositions may be used to treat or prevent coronary artery disease.

In addition to coronary artery disease, oxidative damage also contributes to the late stage complications of diabetes, cataracts, nephropathy (damage to the kidneys), and neuropathy (nerve damage) scar. Due to the increased oxidative stress, these patients typically have higher leverls of oxidized lipids in their blood and abnormally low levels of anti-oxidants. Their Vitamin C levels are often so low that some late stage diabetics are close to suffering from scurvy. High dose anti-oxidants are clearly indicated. Thus, in one embodiment, the presently disclosed compositions may be useful to treat or prevent any of these conditions.

Cataract and age-related macular degeneration (damage to the retina) are the two leading causes of blindness in developed countries. In the USA, cataract surgery is the single largest item in the Medicare budget, costing some $ 3.2 billion per annum. Worldwide, cataracts blind 50 million people every year; but the real tragedy is that much of this is preventable.

The risk of cataracts is increased by oxidative stresses such as increased exposure to UV, which oxidizes the normally transparent proteins in the lens of the eye, and possibly smoking. This risk is reduced by anti-oxidants such as vitamins C and E, alpha-lipoic acid, turmeric, and possibly beta carotene.

It is estimated that 30-50 per cent of all cases of cataracts could be prevented by eating more anti-oxidants. In addition, adding a supplement of riboflavin may boost the metabolism of glutathione, an important anti-oxidant in the eye. A half an aspirin or a spoonful of turmeric may also be added to protect lens proteins from destructive glycosylation reactions (glycosylation results in the cross-linking of proteins.)

The presently disclosed composition may also be useful to treat macular degeneration, which is the other major cause of deteriorating sight. Here again, oxidative damage is involved, and anti-oxidants such as vitamins E, C and beta carotene are protective. This is probably not the most effective combination of anti-oxidants, however. In primates, the main anti-oxidants in the retina are the carotenoids lutein and zeaxanthin. The optimal strategy to preserve sight, even if it has started to fail, is preferably a combination of lutein and zeaxanthin, together with Vitamin C, riboflavin, lycopene, selenium and a turmeric supplement to boost the anti-oxidant enzyme glutathione peroxidase; and the flavonoids in bilberry, a herb traditionally used to treat visual complaints. Thus, the presently disclosed compositions may comprise one or more of these compounds in treating macular degeneration and other age-related sight problems.

The presently disclosed compositions may also be useful to treat Parkinson's Disease. In Parkinson's Disease, toxic tetrahydroquinolones are found in the affected areas of the brain. These compounds are mitochondrial toxins that cause a decrease in glutathione, an increase in free radical formation, and an increase in nerve membrane damage and death. Livestock may also suffer from a similar condition. For example, if a horse is unlucky enough to eat yellow star thistles, which contain compounds called sesquiterpene lactones, the horse may develop a condition rather like Parkinson's Disease.

If the changes in mitochondrial function and anti-oxidant status are an important part of the disease process in Parkinson's Disease, a nutritional approach may be effective. Thus, in one embodiment, the presently disclosed composition comprises a wide spectrum of anti-oxidant compounds (plus Co-enzyme Q10 and beta carotene) to try to improve mitochondrial function; cysteine and alpha-lipoic acid to help boost glutathione levels; and flavonoids to bind free iron that is released when cells in the brain die and triggers the production of more free radicals. The presently disclosed compositions may comprise one or more of these compounds.

The presently disclosed compositions may also comprise flavonoids. Hawthorn flavonoids are a particularly useful group of flavonoids. These powerful anti-oxidants enter the brain (hawthorn is known to cause sedation) and probably enter and protect nerve cell membranes from oxidative damage. These compounds may be further combined with thyme oil, or thymol, which has a similar effect. There is also some evidence suggesting that nitrous oxide (NO) radicals may be involved in the disease; so turmeric, together with beta carotene, or the even more effective carotenoid lycopene, are also worth incorporating into some embodiments of the presently disclosed compositions.

Finally, Vitamin E may be added to this multi-component composition. Vitamin E, by itself, may not be very helpful in treating this condition. But chronic Vitamin E depletion is linked to a form of brain damage in animals rather like the damage found in Parkinson's Disease. In addition, in humans who cannot absorb Vitamin E, the risk of Parkinson's Disease rises.

Something rather similar to Parkinson's Disease is found in patients given long-term anti-psychotic medication. They often develop a syndrome, called tardive dyskinesia (TD). Free radical damage is thought to be involved in this syndrome. Some scientists report that large doses of Vitamin E help, although the evidence is disputed. Vitamin E is an important anti-oxidant in the brain, but at least six months of high-dose supplementation are needed to bring the levels up to where they are needed. By that time, much of the nerve damage caused by the anti-psychotic drugs may have already occurred. Thus, regular intake of anti-oxidants, as provided in the presently disclosed compositions, may help prevent or treat this condition.

The presently disclosed compositions may also be useful to treat or prevent asthma. Cases of asthma are doubling every 10 years. This astonishing trend has generated much research into possible causes of the disease, such as atmospheric pollution, excessive hygiene in childhood, or exposure to the house dust mite—but no definitive cause has been proven.

Some work links asthma to car exhaust fumes. Diesel fumes in particular have been shown to lead to the formation of dangerous NO (nitrous oxide) radicals and anti-oxidant depletion. It is unlikely that this is the sole causative agent, however, because, e.g., although Stockholm has much cleaner air than London, their asthma problem is just as bad.

Nutritional factors are also an important factor in asthma. For example, recent surveys show that children in the Mediterranean countries are relatively unlikely to get asthma. In addition, decreased anti-oxidant consumption may well make asthmas more likely. Vitamin C is important in protecting the lungs from oxidative damage: a high vitamin C intake is linked to better lung function, even in smokers and in patients suffering from chronic obstructive lung disease.

The presently disclosed composition may also comprise fish oil. Fish oil reduces inflammation of the airways in high doses (8-10 g/day) and is usually combined with anti-oxidants, preferably vitamins E and C and flavonoids. These will reduce the formation of LOPs (Lipid Oxidation Products) that may contribute to the inflammation of the lungs, which underlies asthma.

The presently disclosed composition may comprise magnesium. A high magnesium intake is linked to improved lung function, and some clinicians have found magnesium aerosols useful in relaxing the airways of their asthmatic patients.

The presently disclosed composition may be used to treat arthritis. Low anti-oxidant consumption is a risk factor for developing arthritis. Some anti-oxidants, such as beta carotene, reduce symptoms in animal models of arthritis. Other anti-oxidants, such as those found in ginger, reduce joint swelling and pain in clinical trials, but these flavonoid compounds have specific anti-inflammatory properties.

Tumour necrosis factor alpha (TNF-alpha) is important in inflammatory conditions such as asthma, Crohn's disease, and arthritis. In fact, anti-TNF antibodies have been used with some success in clinical trials of arthritis. The success, however, was tempered; patients that made antibodies to the anti-TNF antibodies developed allergic reactions.

A nutritional approach may offer a more effective solution. For example, the spice turmeric contains curcumin, a powerful inhibitor of TNF-alpha. A combination of turmeric and ginger, which blocks the key inflammatory enzymes, also has powerful anti-arthritic properties, when combined with a high dose (i.e., 8-10 g per day) fish or hemp oil, 1200 mg per day of vitamin E, and 1-2 g per day of vitamin C.

The presently disclosed compositions may also be used to treat or prevent tissue disorders, particularly on the skin and in the digestive tract. Free radicals are involved in the destruction of tissue that leads to ulcers of the skin and digestive tract. Smoking, which decreases our anti-oxidant defenses, is a risk factor. Anti-oxidants can treat these conditions and, if used prophylactically, may prevent them.

The flavonoids may be particularly useful in treating or preventing these tissue disorders. The flavonoids are one of the most important groups of compounds derived from plants. Over 20,000 have now been identified since the early days when they were all lumped together as vitamin P by the great Hungarian biochemist Szent Gyorgyi in 1936 (who also discovered vitamin C).

Following Szent Gyorgyi's discovery, pharmaceutical companies brought out a range of medicines containing vitamin P but, by the '60s, most had disappeared. As a natural compound, nobody knew exactly what vitamin P was, how best to measure it, or even whether it was a single compound or a group of compounds. Thus, the drug companies found it difficult to produce a reliable product.

Modern techniques have resolved these problems. The flavonoids have been identified and divided into approximately 12 sub-types, many of which have profound anti-oxidant activity. Each of these groups is under intensive study. Interestingly enough, the results often mirror long-established folk medicine. For example, it has been discovered that one group of flavonoids, especially good at mopping up damaging free radicals in the liver, is found in particularly high levels in milk thistle, which is a herb traditionally used to treat liver disease. Another type of flavonoid which quenches free radicals in the lining of arteries occurs in very high concentrations in hawthorn and yarrow, two plants that have long been used to treat cardiovascular disease.

These are not coincidences. While not all herbal remedies are effective, many remedies have been proven through generations of experience dating back thousands of years. Very often, the herbal lore in different countries, and even different continents, uses the same herbs and plants to treat the same diseases. Again this is no coincidence.

So it is not entirely surprising that when the tools of modern science are brought to bear on traditional herbal remedies, they uncover a wealth of valid medical information—information which is forming the basis for new, rational, and often highly effective forms of preventative and curative medicine.

The key to the role of flavonoids is that many of them are extremely potent anti-oxidants and anti-flammatory agents. Different flavonoids work in different tissues of the body; some can enter the brain, for instance, whereas others appear to concentrate in the lining of blood vessels. This means that different flavonoids can be used to target different tissues. For example, a flavonoid which is taken up by the lining in the capillaries might be expected to be good for capillary function. Ginkgo flavonoids, widely used to improve blood flow to the brain, hands and feet, are absorbed in this manner.

Many flavonoids neutralize free radicals, including the highly dangerous hydroxyl radical. One group is particularly good at quenching the radicals which cause liver damage (these are the flavonoids found in milk thistle).

The presently disclosed compositions may also comprise phytate. Phytate is known to act as an anti-oxidant. It is generally regarded as an anti-nutrient because it binds iron. However, in certain circumstances this is a good thing, as excess (free) iron in the body is a potent source of free radicals, and a potential carcinogen. Thus, when phytate binds iron, it is effectively acting as an anti-oxidant. This helps to explain why phytate is a powerful inhibitor of colon cancer, where free iron is one of the key causative agents. (Phytate is not recommended for everyone, however. Iron deficiency (anaemia) is widespread, particularly among women of child-bearing age, and too much phytate could complicate an existing anemic condition.)

The presently disclosed compositions may also comprise phytosterols, which protect against carcinogenic bile acids. Phytosterols are the plant equivalents of cholesterol. They are poorly absorbed and remain in the gut, where they are thought to protect against the harmful effects of certain (secondary) bile acids. These bile acids are formed from cholesterol and have mutagenic and carcinogenic properties. This may be why some phytosterols are capable of reducing the incidence of colon cancer by as much as 50 per cent.

The presently disclosed compositions may also comprise saponins, which possess anti-mutation, anti-oxidant properties. Saponins are anti-oxidants because they protect against free radical damage. Lab tests have also shown that saponins prevent mutations that can lead to cancer.

The presently disclosed compositions may also comprise phenolic compounds that protect DNA. Phenolic compounds also possess anti-oxidant activity, and are thought to protect DNA from attack by certain categories of carcinogens.

The presently disclosed compositions may also comprise isoflavones. Isoflavones block estrogen, a hormone linked to an increased risk of breast and other hormone-dependent cancers. They act similarly to Tamoxifen, a drug widely used to treat and prevent breast cancer.

Prostate cancer, like breast cancer, is usually hormone-dependent. But while breast cancer is thought to be stimulated by estrogen, prostate cancer is often stimulated by testosterone. Isoflavones, which block testosterone, reduce the tendency for prostate cancers to grow.

Some flavonoids bind to dangerous free iron and copper in the body, thereby stopping free radical formation. Many are capable of locking up free oxygen and preventing the oxidation of ascorbic acid, thereby protecting vitamin C in fruit, fruit juices, and in the body.

One flavonoid, quercitin, found in onions and apples may be one of the most cardio-protective substances yet discovered. The Zutphen Elderly Study measured the flavonoid content of the participants' diets and discovered that the number of cardiac deaths in the group eating the most flavonoids was only a quarter of the death rate of the group which ate the least flavonoids. And quercitin accounted for two-thirds of the total flavonoid intake. In fact, all mortality rates were lower in the high flavonoid group, even when other dietary anti-oxidants such as vitamins C and E were ruled out. Since Zutphen, two more trials have found that quercitin (a powerful anti-oxidant and anti-inflammatory agent) is cardio-protective.

There has been some controversy over whether quercitin is absorbed from the gut, because little is found in the bloodstream. However, recent work shows that quercitin and related compounds are absorbed and concentrated elsewhere in the body. There's other evidence that flavonoids are absorbed. Tannins, for example, which consist of long chains of flavonoids linked together, protect against stroke in hypertensive animals. Tannins are found in, e.g., tea, wine, quince and persimmons.

Procyanidins are an extremely promising group of flavonoids. They are well absorbed from the gut, and are already used in the form of Pycnogenol (a commercial preparation of bioflavonoids that includes catechias, phenolic acid, proan, and cyanidins) and grapeseed extracts to treat arthritic conditions, because of their ability to quench free radicals and stop the breakdown of synovial (lubricating) fluid inside inflamed joints. These flavonoids also target blood vessels and, once there, protect the connective tissue in the artery walls by exerting a powerful anti-oxidant, anti-inflammatory and anti-permeability effect. They also block enzymes which have a destructive effect on the connective tissues.

These protective actions from the high procyanidin content of black grapes (and red wine) are probably one of the main factors underlying the so-called French paradox. The French eat a high fat diet, yet are relatively immune from heart disease. There are probably several factors involved, including the widespread use of olive oil. However, there is evidence that consumption of two to four glasses of red wine a day reduces the risk of a heart attack by an astonishing 40 per cent.

Thus, the presently disclosed composition may comprise a combination of querticin and a procyandin compound (e.g., grapeseed extract). Such a combination may be used to reduce the risk of heart attacks.

These two types of flavonoids, though similar, appear to block two distinct steps in the chain of events that leads to atheroma formation. Quercitin's main role is to protect lipids in the blood from oxidizing; whereas the procyanidin's main role is to prevent oxidative damage from occurring in the blood vessel wall.

The procyanidins' ability to bind to and protect the fibers in the vessel walls (such as collagen and elastin) from oxidative or enzyme attack helps to reduce the amount of damage to the walls. If there is already damage, the two flavonoids seem to stop the site from becoming inflamed and slow the furring of the arteries. Procyanidins are best used preventatively, but even after a heart attack they can help. Their phenomenal ability to scavenge free radicals means that a procyanidin product will reduce your risk of developing a life-threatening arrhythmia after the attack.

Because of the ability of procyanidins to keep blood vessels healthy, it's not surprising that reports have appeared describing improvements in varicose veins, oedema and haemorrhoids. Thus, the presently disclosed composition may be used to treat any of these conditions.

Anyone suffering from any of these should generally take a procyanidin product for at least two months, although in some cases improvement is noticeable in one month. Vitamin C is preferably taken with it, and in serious cases an additional glucosamine supplement may be used.

Procyanidins may also be used to treat sight loss in diabetics, which is caused by the growth of new blood vessels supplying the retina. The procyanidins block this effect. Other flavonoids can help diabetics too. For example, the cholesterol in blood is more prone to oxidation than cholesterol in non-diabetics. This is one reason why diabetics suffer more heart attacks. In a recent study, a flavonoid preparation (Diesmin) not only reduced the rate of cholesterol and lipid oxidation, but also reduced the rate that proteins were damaged (cross-linked) by the excess blood sugar. This would not only reduce blood vessel damage in the eye and elsewhere in the body, but also protect against cataracts and renal damage, where excess cross-linking is involved.

Procyanidins are being incorporated into the latest cosmetics. They form a protective shield around the collagen fibers which give skin its firmness and texture, and protect them against the enzymes which break down these fibers. They also protect against free radical damage.

The development of these cosmetic formulations is still in the early stages, but procyanidin's anti-allergic, anti-inflammatory and anti-oxidant properties may constitute a major cosmetic break-through, especially when combined with other anti-aging nutrients such as the amino sugars. Thus, the presently disclosed compositions may be incorporated into a cosmetic formulation.

Procyanidins target the bacteria which cause dental decay. The flavonoids seem to stop the bacteria from sticking to the teeth and dental plaque. Some dental scientists are looking at these flavonoids as a way of slowing down tooth decay. The anti-inflammatory effects may also help to control or minimize gingivitis (gum disease) which is responsible for more tooth loss than dental decay. Thus, the presently disclosed compositions may be incorporated into dental treatments.

Flavonoids boast an extensive array of anti-cancer effects which provide for an extremely impressive cancer defense. Free radicals damage DNA. Many flavonoids are potent anti-oxidants which mop up large numbers of free radicals, and reduce the amount of DNA damage. Free radicals also damage cell membranes. This type of damage may lead to tumor formation. Thus, anti-oxidants like flavonoids should help prevent or reduce such effects. Not surprisingly, a great deal of nutritional research is concentrating on just this area. Some of the most active and best researched compounds, along with some exemplary sources of the compounds, include:

quercitin—onions;

ellagic acid—walnuts, pecan nuts;

caffeic acid—coffee beans;

chlorogenic acid—tomatoes;

epigallocatechingallate—tea;

carnosic acid—rosemary; and

genistein—soy.

The present composition may also comprise protease inhibitors (e.g., lectins), which block genes that promote cancer. Soy beans are a source of these protease inhibitors, which possess growth-inhibiting properties that reduce the spread of cancer. Recent work has demonstrated that protease inhibitors also block the action of a number of genes which cause cancer. Most protease inhibitors are destroyed by cooking, but there is evidence that enough survive to confer a significant protective effect.

King of the isoflavones, and subject of well over 300 research papers to date, is genistein. Genistein has little effect on normal cells, but is a powerful inhibitor of nearly every cancer cell type examined so far. The broad spectrum anti-cancer effects of genistein are attributed to its profound mode of action: it inhibits several of the products of oncogenes, which are genes that cause cancer.

The presently disclosed composition may contain soy, which is also useful to protect against heart disease, such as coronary artery disease. One of the most insidious aspects of coronary artery disease (CAD) is that it is a hidden disease. For most people, the first sign that anything is wrong is the first heart attack. The great majority of survivors are left with a permanently damaged heart and a long-term risk of complications.

Even advanced cases of CAD are often not diagnosed in time. An American investigation called the Sudden Death Study, discovered that an astonishing one in four people who died suddenly of a heart attack had seen their doctor in the week before they died. But they had not been diagnosed accurately, and had not been hospitalized. This is why with coronary artery disease, as in so many other diseases, prevention is better than cure.

Diet is the key. A diet rich in animal fats and low in anti-oxidants and fish oil is a fast route to a heart attack, as is smoking. And so is high blood cholesterol. The presently disclosed compositions are useful to provide the components needed for a healthy diet.

Bacterial flora in the body are also an important component of optimal health. The large bowel, where the majority of gastrointestinal cancers occur, contains four to five hundred different species of bacteria, known in medical language as ‘flora’. Some of these can cause serious illness, while others are associated with positive health. Since the beginning of this century, doctors have experimented with different diets in an attempt to modify the gastrointestinal flora, and push it in a ‘healthy’ direction (without much success).

There are at least two types of health-promoting bacteria, the lactobacilli and the bifidobacteria. Some of these are found in yogurt, and various scientists and nutritionists have used yogurt to try to change the flora of the lower bowel.

However, the bacteria have a limited shelf-life, even when freeze-dried; and many of them are unable to survive the acid conditions in the stomach. Moreover, even if the bacteria do arrive in the colon, they have to compete with the dense population of hostile bacteria that are already established there. As long as you eat a daily helping of yogurt, some lactobacilli and bifidobacteria remain in the gut, but they disappear almost immediately when the yogurt diet stops.

Prebiotics have none of these disadvantages. They are stable, safe (found in many staple foods), and have a longer-lasting effect on the gut's bacterial flora. They promote the growth of ‘healthy’ bacteria, and inhibit other bacteria which can cause disease by overgrowth or by producing toxins. Thus, the presently disclosed compositions may comprise prebiotics.

Two of the main types of natural pre-biotics are insulin and oligo-fructose. The general rule is that the fresher the vegetable, the higher its insulin content. When plants such as onions are stored for long periods of time, and particularly in cold storage, their pre-biotic content declines dramatically. Because most people buy fruits and vegetables from supermarkets where the foods may have been in cold storage for months, their pre-biotic intake may actually be very low. A low intake of pre-biotics leads to increased numbers of disease-causing bacteria in the gut—which could be the cause of many gastrointestinal and other health complaints.

Unlike most sugars and starches, pre-biotics cannot be digested and they pass into the colon intact. Once there, they act as a growth enhancer for the health-promoting lactobacilli and bifidobacteria. As the ‘good’ bacteria multiply, they secrete enzymes which break down pre-biotics into acids, such as acetic and butyric acid. These inhibit the growth of disease-causing bacteria. The ‘good’ bacteria also secrete antibiotic substances which restrain the ‘unhealthy’ bugs, including most of those responsible for food poisoning.

As a result of ingesting pre-biotics, the balance of the bacterial flora in the gut tips in a healthy direction. The flourishing lactobacilli and bifidobacteria in the gut join gastric acid, the digestive enzymes, and the immune system in ‘crowding’ out disease-causing bacteria. Such individuals benefit from improved intestinal ‘regularity’ and an increased resistance to food poisoning. Such conditions are generally reported to be present in the gut of breast-fed infants, but only in a mere 25 per cent of bottle-fed infants. This explains why breast-fed babies are more resistant to stomach upsets and diarrhoea. Accordingly, recent studies show that live yogurt cultures fed to infants significantly reduce their risk of contracting diarrhea, and speed recovery when given as a treatment for diarrhea.

As we age, the proportion of bifidobacteria and lactobacilli relative to the other bacterial flora gradually falls. This is one reason why we become more prone to gastrointestinal upsets. It is probably also linked to the age-related increase in the risk of bowel cancer and other illnesses.

The risk of colon cancer is also increased by a diet high in animal fats—a diet which causes more bile to be secreted. Unhealthy gut bacteria convert bile acids into cancer-causing compounds that increase the risk of liver cancer. Pre-biotics reduce the disease-causing bacteria in the gut, and the amount of cancer-causing compounds they produce to yield a protective effect.

Finally, as the prebiotics grow they bind free iron, thereby reducing levels of free radicals in the colon. This provides another cancer-preventing property.

As indicated on the list of ingredients on a carton of yogurt, there are significant amounts of thiamin, riboflavin and other vitamins. This is because the lactobacilli and bifidobacteria make B vitamins, and are probably the major species of bacteria in the colon which do this.

Vitamin B depletion is surprisingly common in developed countries. This deficiency is a major risk factor for coronary artery disease. So pre-biotics, which promote the good bacteria in the gut and the levels of vitamin B, may be used as a cardio-protective compound. It is believed that this is achieved by lowering homocysteine, and simultaneously raising HDL levels.

This is one way in which fats and insulin contribute to a healthy heart; although LDL cholesterol reduction also plays a role. Short chain pre-biotics (i.e., FOS) are rapidly fermented, stimulating the production of bifidobacteria (bifidogenesis) in the proximal colon. As they grow they bind the bile acids present in this part of the gut and remove them from the body. This lowers LDL cholesterol levels and confers additional cardio-protection. The combination of bifidogenesis and bile acid binding is also likely to be cancer protective, especially if FOS is combined with longer chain pre-biotics.

The edible fats and oils, known collectively as fatty acids, are basically similar compounds. Oils, however, melt at lower temperatures than fats, and at room temperature oils are liquid and fats are solid. Fatty acids (fats and oils) are a rich source of calories, which can either be ‘burned’ to produce energy, or stored as fat for lean times ahead. They are also incorporated into cell membranes and other tissues, where they have an important structural role. Finally, fatty acids are metabolized into compounds called eicosanoids. Fats and oils produce quite different eicosanoids: broadly speaking, fats form eicosanoids that increase inflammation, and oils produce eicosanoids that reduce inflammation.

This difference is important, because many chronic diseases are basically inflammatory conditions. These include arthritis (inflammation of the joints), eczema (inflammation of the skin), asthma (inflammation of the lungs), and coronary artery disease (inflammation of the arteries).

An individual's risk for these conditions is affected by their genetic make-up, tobacco consumption, and the amount of anti-oxidants in their diet. But the fats and oils in the diet are also important.

Saturated fats raise the levels of ‘bad’, i.e., LDL cholesterol in the blood. Fish oil, on the other hand, slows the formation of LDL cholesterol, reducing the risk of heart attacks. The medical profession found it hard to believe that a simple switch from fats to oils could have significant health benefits. However, the weight of all the clinical trials that have demonstrated positive health benefits from such a switch has begun to change their opinion. The evidence is particularly stong in the area of coronary artery disease, where the poly-unsaturated fatty acids (PUFAS) are rapidly gaining a role in even conventional circles.

Not all PUFAs are the same. Some of them are more important than others; and a few are so important that they are termed, collectively, the Essential PUFAs.

There are two families of essential poly-unsaturated fatty acids: Omega 6 and Omega 3, both of which are oils. These oils are vital for the functioning of every cell in our bodies, and yet our bodies cannot make them. We have to obtain them from our diet. Thus, in that sense they are similar to vitamins. Once the oils have been absorbed from our food, our enzymes make all the other Omega 3 and 6 PUFAs our cells and systems need.

PUFAs are a powerful force for good health, but as with other powerful agents, they should not be taken indiscriminately. The ratio of the various PUFAs in the diet is important. In most mammals' cells, the level of Omega 6 (from vegetable sources) is three to four times higher than the Omega 3 content (from fish sources), which gives us a standard as to what the optimal ratio of the two should be.

The problem with poly-unsaturated fatty acids (PUFAs) is that they are very prone to going rancid, or oxidizing. It's preferred to eat a high PUFA diet from unprocessed foods such as nuts and grains, because these foods contain their own anti-oxidants, such as vitamin E, carotenoids and flavonoids. Without these anti-oxidants, they would go rancid, and the seeds, grains and nuts would not survive long enough to propagate the species.

However, PUFAs from refined poly-unsaturated oils and spreads could cause unwanted problems. These processed foods have had the naturally occurring anti-oxidants stripped away, and are therefore highly prone to being oxidized. This has two potentially very serious effects. First, the PUFAs form lipid oxidation products (LOPs) that are extremely toxic. LOPs literally rip holes in the lining of the arteries and are therefore a substantial risk factor for heart disease. Second, PUFAs soak up anti-oxidants in the body as they are oxidized, leaving an excess of free radicals and causing accelerated aging. The end result is an increase in the risk and severity of chronic degenerative diseases ranging from heart disease to cancer to asthma.

At the Royal Prince Alfred Hospital in Sydney, Australia, epidemiologists have linked the huge increase in childhood asthma with a five-fold increase in margarine consumption since the War. Their theory is that the increased PUFA content in the diet has led to an increase in inflammatory and toxic LOPs, which leave the airways raw, and trigger the asthma.

Since poly-unsaturated oils are so prone to oxidation, it is preferable to combine them with an anti-oxidant preparation, especially for smokers. Otherwise, the load of PUFA radicals and oxidation products will increase in the body, which will be harmful to the arteries. This need for anti-oxidants may explain the disappointing results of a recent trial, which found that fish oil supplements on their own have little effect on preventing atheroma formation.

In one embodiment, the present compositions comprise a combination of anti-oxidant compounds including, but not limited one or more of the following:

Vitamin E (400 IU/day);

Mixed flavonoids (100-500 mg);

Vitamin C (500-1000 mg);

Co-enzyme Q10 (30-120 mg/day); and

Mixed carotenoids (10-20 mg).

The presently disclosed compositions may comprise Co-enzyme Q10, which is particularly preferred. Apart from its other known benefits, it is very good at preventing the increase in free radicals otherwise caused by fish oil supplements.

Co-enzyme Q10 is often referred to as Vitamin Q, but although it is vital to life, and occurs in trace amounts in certain foods (such as sardines), Q10 is not technically a vitamin because it is produced in small amounts in the liver. Unfortunately, the process requires at least six other vitamins and minerals; and most people are depleted in one or more of these nutrients. Heavy drinking and liver disease slow the production of Q10 even further. Moreover, after the age of about 40, the ability to make Q10 declines; and levels of Q10 in the diet are too low to compensate for this loss of Q10 production.

The presently disclosed compositions may comprise one or more amino sugars. Amino sugars are among the most important building blocks in the body. Dr. Frances Burton, an expert in amino sugars at the University of British Columbia, expressed it beautifully, stating, “Amino sugars make up the structure of all tissues, on the surface of cells and in the spaces in between them; forming the substance which binds cells together, the membranes which envelop them and the protective layers which cover them.”

Macro-molecules built up from amino sugars, called GAGs and PGs, together with the proteins called collagen and elastin, make up the framework for all our tissues. Combined in different proportions, they make tissues soft, slippery, squashy, stretchy or strong. They give our bodies shape, organization, definition and function. GAGs and collagen make up tendons, ligaments, heart valves, skin and finger-nails. Combined with another protein, elastin, they make cartilage in joints and the discs in the spine.

All of these macro-molecules are constantly being broken down and replaced as part of the body's on-going general maintenance programs. How fast this happens is crucial in maintaining the strength and elasticity of every tissue in the body. However, there is one major problem when the amino sugars are broken down to quickly or the body's stores of these molecules are depleted. This can cause arthritic and other inflammatory conditions, as discussed below.

Based an our current understanding of arthritis, a nutritional program which combines glucosamine with natural antiinflammatory flavonoids may be used to greatly reduce the symptoms and the risk of the disease. Thus, the presently disclosed compositions may be used to treat or prevent arthritis.

This is likely to produce the best results in the elderly, who are more depleted in glucosamine and anti-oxidants, such as flavonoids. Such nutritional program could halt, or even reverse the progress of arthritis.

The present compositions may also be used to treat or prevent other inflammatory conditions. For example, the cells lining the gut have a very high turnover rate. In chronic inflammatory conditions, the rate is even faster. In these conditions, the rate of cell growth may outstrip the rate of glucosamine and GAG (glycosaminoglycan) production. In fact, the inflammation itself may inhibit the production of GAGs, and increase the rate at which they are broken down.

Patients with active inflammatory bowel disease (including Crohn's Disease and Ulcerative Colitis) have very low levels of GAGs in their intestinal walls. Such a depletion would be expected to cause local vascular problems, such as increasing leakage of fluid into the surrounding tissues, and contribute to several distinct types of local tissue damage that are all found in chronic inflammatory bowel disease.

Low levels of amino sugar compounds cause other problems. Such a depletion would eventually affect the thin but vital glycolipid layer which protects the intestinal wall. Since the gut, more than any other organ, is constantly challenged by bacteria, viruses, digestive juices and dietary antigens, losing this vital protection could lead to health problems, including food allergies, which are thought to occur in conditions where the gut wall is abnormally permeable.

In the exposed skin of the face and hands, much of the aging is caused by free radicals liberated by sunlight. Free radicals damage collagen and elastin fibres in the skin, and the GAGs and PGs. This damage to the extra-cellular matrix leads to a loss of firmness, plumpness and elasticity, and is a large part of the skin aging process.

The amino sugar compounds in the skin are constantly being broken down and replaced. As much as one fifth of the glucose in the blood is destined for connective tissue formation. But if the glucosamine-producing enzymes slow down, as they do with age, they cannot keep pace with the deterioration caused by exposure to ultra-violet light (UV), cigarette smoke, pollution, and other sources of free radicals. The connective tissue that gives the skin strength, elasticity and firmness deteriorates, with all-too-obvious results.

To protect the skin, sugars such as glucosamine, vitamin C, and zinc are needed for collagen and elastin synthesis. Maintaining the extra-cellular matrix requires an antioxidant mix containing the procyanidin flavonoids (e.g., bilberry or grapeseed), which concentrate in the micro-fibres of connective tissue and protect them from free radical damage; mixed carotenoids have a similar effect.

The presently disclosed composition may also comprise one or more anti-glycosylants. Glycosylation (the attachment of sugar molecules) of collagen and elastin increases with aging. This disrupts the connective tissue by a process known as cross-linking. This has the unfortunate effect of leaving the skin less elastic, less permeable and more prone to wrinkles.

Half a tablet of aspirin helps prevent glycosylation, as does a tablespoon of turmeric. Vitamin C has a similar effect, and is another essential part of an anti-aging program; especially as it is essential for the synthesis of the skin protein collagen. Thus, the presently disclosed composition may comprise one or more of these compounds. In one embodiment, the composition may be used to combat the effects of aging.

The presently disclosed composition may comprise one or more silicic acid supplements. High levels of aluminium damage the fibroblasts and other cells responsible for building and repairing the extra-ceflular matrix, in the skin and elsewhere. Silicic acid is the most effective shield against ingested aluminium, and can enhance the regeneration of the extra-ceflular matrix.

The presently disclosed composition may comprise one or more methyl groups. Methyl groups, like vitamins, are essential in our diet. Foods that contain significant levels of the methyl groups are, in descending order, sugar beet, sugar cane, prawns, shrimps, and eggs. To give an idea of the importance of methyl groups, the nervous system, the immune system, the heart and blood vessels, the kidneys, and the liver all depend on methyl groups to function normally.

A diet low in methyl groups damages all the above systems. Stress becomes more destructive, toxins become more toxic, carcinogens more carcinogenic. In fact, a lack of methyl groups in the diet is the only dietary deficiency known to be directly carcinogenic. If there are not enough methyl groups, DNA synthesis can go awry, leading to the activation of oncogenes (cancer-causing genes).

Methyl group deficiency in the diet is so dangerous because of the importance of a process called the methyl group cycle, discussed briefly below.

Methyl groups are a simple combination of carbon and hydrogen atoms. Methyl groups from the diet combine with homocysteine in the body to form methionine. Methionine is then turned into S-adenosyl methionine (SAM). SAM is used in the body to produce many essential compounds. These include creatine and carnitine (important in energy production), phospholipids (essential molecules involved in-cell membrane and especially nerve health), RNA and DNA, the stress hormones epinephrine and nor-epinephrine, and the neurotransmitters involved in mental states. Methyl groups are also essential to the basic functioning of the immune system.

If there are inadequate methyl groups in the diet, any of these functions may be impaired. But there is a further serious complication from insufficient methyl groups in the diet. After SAM has donated its methyl group it becomes S-adenosyl homocysteine, which breaks down into the toxic amino acid homocysteine. If there are too few methyl groups from the diet to transform this homocysteine back again into methionine, levels of homocysteine rise. This increases the risk of cardiovascular disease and Alzheimer's disease.

The body cannot synthesize methyl groups. Thus, a constant dietary intake of methyl groups is essential to maintain the cycle, in order to keep levels of SAM up and levels of homocysteine down. The principal dietary sources of methyl groups are, in descending order, the nutrients betaine, chaiine, and methionine; and to a lesser extent the vitamins B6, B12, and folic acid. Although the vitamins provide methyl groups, they are not the best donors; betaine is more effective. The presently disclosed compositions may comprise one or more methyl donating compounds.

Under conditions of stress (such as disease), the need for methyl groups increases. This is because methyl groups are needed for the formation of stress hormones, for various defense mechanisms and for the synthesis of polyamines (e.g., RNA and DNA), all of which are needed for tissue repair. When stress increases the demands for methyl groups, the resulting shortfall in methyl groups inevitably leads to an increase in homocysteine—another reason why stress has a negative impact on health.

As an excellent methyl group donor, betaine is very effective at lowering levels of homocysteine. Most humans, however, do not consume much betaine; and in this situation, the B vitamins become the next line of defense. One recent study found that people who ate high levels of folic acid (a B vitamin) were 69% less likely to suffer a fatal heart attack than those whose diet contained low levels of folic acid.

Sadly, B vitamin deficiency is all too common. This may explain why excessive levels of homocysteine (with its corresponding increase in the risk of heart disease and Alzheimer's disease) are so typical.

A preparation containing a spectrum of B vitamins reduces levels of homocysteine. This is why supplements of folic acid and Vitamins B6 and B12 are increasingly being used to reduce homocysteine levels, and the risk of homocysteine-related cardiovascular and neurological diseases.

There are several known mixtures containing vitamins, minerals, and other substances that are used as food supplements and exhibit health promoting effects. These mixtures usually contain substances in unspecified amounts, thereby causing unwanted side effects. Some of these mixtures are used to effect health in a general manner. However, such mixtures are less effective in cases where a specific disease needs to be treated or prevented. The present invention overcomes these deficiencies. The presently disclosed compositions are defined mixtures of compounds useful as food supplements or medicaments to treat or prevent specific diseases.

The presently disclsosed compositions may be used to treat or prevent a number of diseases including, but not limited to, mental decline, diabetes, arthritis, osteoporosis, asthma, and age-related eye disease (in particular macular degeneration). These presently disclosed compositions may be provided as food additives or as medicaments.

The effects of nutraceutical intervention are always filtered through the nutritional baseline of the subject or by other lifestyle factors such as smoking and activity levels or by genetic variations. One cannot change genetic variations. But in order to achieve optimal results, the neutraceutical intervention is preferably combined with one or more of the following: a reduction or cessation in smoking, a reduction in sodium intake, and an increase in activity levels.

The present invention is directed to compositions comprising mixtures of compounds comprising one or more of the following compounds in the following amounts, wherein not all of them are contained in a single mixture:

a) 800 mcg (2665 IU) of Vitamin A, 500 mg of Vitamin C,15 mcg of Vitamin D, 200 IU of Vitamin E, 200 mcg of Vitamin K,

b) 10 mg of Beta carotene, 6 mg of Lutein, 5 mg of Lycopene, 100 mcg of Zeaxanthin,

c) 7.5 mg of Vitamin B1, 7.5 mg of Vitamin B2, 15 mg of Niacin, 15 mg of Pantothenic acid, 7.5 mg of Vitamin B6, 200 mcg of Folic acid, 6.75 mcg of Vitamin B12,

d) 200 mcg of Selenium (preferably in the yeast form), 10 mg of Zinc, 100 mg of Calcium, 50 mg of Magnesium,120 mcg of Chromium, 2 mg of Copper, 4 mg of Manganese, 100 mcg of Iodine, 100 mcg of Molybdenum,

e) 200 mcg of Biotin, 1000 mg of Betaine, 250 mg of Oligoproanthocyanidins (OPC), 150 mg of Polyphenol complex, 40 mg of Isoflavones (in particular genistein and/or daidzein), 600 mg of Omega-3, 4 to 6 g of Oligosaccharides (FOS) (in particular oligo-fructose and/or beta glucan), 4 to 6 g of insulin, 30-60 mg of Co-Q10, and

f) 1000 mg of Glucosamine.

The presently disclosed compositions may further comprise additional substances for the purpose of stabilization and formulation of the above mentioned compositions.

The amounts listed above for each single compound within the composition define the average amount of each compound. This amount may vary within an upper or lower limit of up to 15% of the listed amount. Such differences may be caused, for example, by variations between sources of the compounds or by methodological variations from weighing or packaging.

In one embodiment, the presently disclosed composition comprises a mixture of compounds comprising at least:

a) 800 mcg (2665 IU) of Vitamin A, 1000 mg of Vitamin C, 400 IU of Vitamin E, 200 mcg of Vitamin K,

b) 10 mg of Beta carotene, 6 mg of Lutein, 5 mg of Lycopene, 100 mcg of Zeaxant,

c) 7.5 mg of Vitamin B1, 7.5 mg of Vitamin B2, 15 mg of Niacin, 15 mg of Pantothenic acid, 7.5 mg of Vitamin B6, 200 mcg of Folic acid, 6.75 mcg of Vitamin B12,

d) 200 mcg of Selenium (preferably in the yeast form), 10 mg of Zinc, 120 mcg of Chromium, 2 mg of Copper, 4 mg of Manganese, 100 mcg of Iodine, 100 mcg of Molybdenum, and

e) 200 mcg of Biotin, 1000 mg of Betaine, 250 mg of Oligoproanthocyanidins (OPC), 150 mg of Polyphenol complex, 1000 mg of alpha lipoic acid, 600 to 5000 mg of mixed Omega 3 and 6, 4 to 6 g of Oligosaccharides (FOS) (in particular oligo-fructose and/or beta glucan), and 4 to 6 g of insulin.

The presently disclosed composition may further comprise additional substances for the purpose of stabilization and formulation of the above mentioned compositions.

The invention refers further to a method of producing the presently disclosed compositions comprising first producing the single compounds by chemical synthesis or isolating them from natural sources, putting the single compounds together in a suitable vessel in relative amounts as to end up with the specified amount for each compound, mixing the compounds together, and adding additional substances for stabilization and/or formulation of the composition.

The invention refers also to the use of the presently disclosed composition as a food additive or medicament for preventing, curing, or improving the symptoms of diabetes.

In another embodiment, the presently disclosed composition comprises a mixture of compounds comprising at least:

a) 800 mcg (2665 IU) of Vitamin A, 500 mg of Vitamin C, 200 IU of Vitamin E, 200 mcg of Vitamin K,

b) 10 mg of Beta carotene, 6 mg of Lutein, 5 mg of Lycopene, 100 mcg of Zeaxanthin,

c) 7.5 mg of Vitamin B1, 7.5 mg of Vitamin B2, 15 mg of Niacin, 15 mg of Pantothenic acid, 7.5 mg of Vitamin B6, 200 mcg of Folic acid, 6.75 mcg of Vitamin B12,

d) 200 mcg of Selenium (preferably in the yeast form), 10 mg of Zinc, 120 mcg of Chromium, 2 mg of Copper, 4 mg of Manganese, 100 mcg of Iodine, 100 mcg of Molybdenum,

e) 200 mcg of Biotin, 5000 mg of Oligoproanthocyanidins (OPC), 150 mg of Polyphenol complex, 600 mg of Omega 3, 100 mg of Co-Q10, and

f) 1000 mg of Glucosamine.

The composition may further comprise additional substances for the purpose of stabilization and formulation of the above mentioned compositions.

The invention refers further to a method of preparing the presently disclosed composition, comprising producing the single compounds by chemical synthesis or isolating them from natural sources, putting the single compounds together in a suitable vessel in relative amounts as to end up with the specified amount for each compound, mixing the compounds together, and adding additional substances for stabilization and/or formulation of this composition.

The invention refers also to the use of this same composition as food additive or medicament for preventing, curing, or improving the symptoms of osteo-arthritis.

In another embodiment, the presently disclosed composition comprises a mixture of compounds comprising at least:

a) 800 mcg (2665 IU) of Vitamin A, 500 mg of Vitamin C, 20 mcg of Vitamin D, 200 mcg of Vitamin K,

b) 10 mg of Beta carotene, 6 mg of Lutein, 5 mg of Lycopene, 100 mcg of Zeaxanthin,

c) 7.5 mg of Vitamin B1, 7.5 mg of Vitamin B2, 15 mg of Niacin, 15 mg of Pantothenic acid, 7.5 mg of Vitamin B6, 200 mcg of Folic acid, 6.75 mcg of Vitamin B12,

d) 200 mcg of Selenium (preferably in the yeast form), 10 mg of Zinc, 100 mg of Calcium, 50 mg of Magnesium, 120 mcg of Chromium, 2 mg of Copper, 4 mg of Manganese, 100 mcg of Iodine, 100 mcg of Molybdenum,

e) 200 mcg of Biotin, 250 mg of Oligoproanthocyanidins (OPC), 150 mg of Polyphenol complex, 40 mg of Isoflavones (in particular genistein and/or daidzein), 100 mg of Co-Q10, and

f) 1000 mg of Glucosamine.

The composition may further comprise additional substances for the purpose of stabilization and formulation of the above mentioned compositions.

The invention refers further to a method of preparing the presently disclosed composition, comprising producing the single compounds by chemical synthesis or isolating them from natural sources, putting the single compounds together in a suitable vessel in relative amounts as to end up with the specified amount for each compound, mixing the compounds together, and adding additional substances for stabilization and/or formulation of this composition.

The invention refers also to the use of this same composition for preparation of a food additive or medicament for preventing, curing, or improving the symptoms of osteoporosis.

The invention refers to a mixture of compounds comprising at least

a) 800 mcg (2665 IU) of Vitamin A, 1000 mg of Vitamin C, 15 mcg of Vitamin D, 400 IU of Vitamin E, 200 mcg of Vitamin K,

b) 10 mg of Beta carotene, 6 mg of Lutein, 5 mg of Lycopene, 100 mcg of Zeaxanthin,

c) 7.5 mg of Vitamin B1, 7.5 mg of Vitamin B2, 15 mg of Niacin, 15 mg of Pantothenic acid, 7.5 mg of Vitamin B6, 200 mcg of Folic acid, 6.75 mcg of Vitamin B12,

d) 200 mcg of Selenium (preferably in the yeast form), 10 mg of Zinc, 100 mg of Calcium, 50 mg of Magnesium, 120 mcg of Chromium, 2 mg of Copper, 4 mg of Manganese, 100 mcg of Iodine, 100 mcg of Molybdenum, and

e) 200 mcg of Biotin, 1000 mg of Betaine, 250 mg of Oligoproanthocyanidins (OPC), 150 mg of Polyphenol complex, 500 mg of curcuminoids, 100 mg of ginko biloba, 600 to 10 000 mg of mixed Omega 3, 60-90 mg of Co-Q10.

The composition may further comprise additional substances for the purpose of stabilization and formulation of the above mentioned compositions.

The invention refers further to a method of preparing the presently disclosed composition, comprising producing the single compounds by chemical synthesis or isolating them from natural sources, putting the single compounds together in a suitable vessel in relative amounts as to end up with the specified amount for each compound, mixing the compounds together, and adding additional substances for stabilization and/or formulation of this composition.

The invention refers also to the use of this same composition for preparation of a food additive or medicament for preventing, curing, or improving the symptoms of asthma.

In another embodiment, the presently disclosed composition comprises a mixture of compounds comprising at least:

a) 800 mcg (2665 IU) of Vitamin A, 500 mg of Vitamin C, 200 IU of Vitamin E, 200 mcg of Vitamin K,

b) 10 mg of Beta carotene, 6 mg of Lutein, 5 mg of Lycopene, 100 mcg of Zeaxanthin,

c) 7.5 mg of Vitamin B1, 7.5 mg of Vitamin B2, 15 mg of Niacin, 15 mg of Pantothenic acid, 7.5 mg of Vitamin B6, 200 mcg of Folic acid, 6.75 mcg of Vitamin B12,

d) 200 mcg of Selenium (preferably the yeast form), 10 mg of Zinc, 120 mcg of Chromium, 2 mg of Copper, 4 mg of Manganese, 100 mcg of Iodine, 100 mcg of Molybdenum, and

e) 200 mcg of Biotin, 1000 mg of Betaine, 250 mg of Oligoproanthocyanidins (OPC), 150 mg of Polyphenol complex, 40 mg of Isoflavones (in particular genistein and/or daidzein), 600 mg of Omega 3, 4 to 6 g of Oligosaccharides (FOS) (in particular oligo-fructose and/or beta glucan), 4 to 6 g of insulin, and 60-90 mg of Co-Q10.

The composition may further comprise additional substances for the purpose of stabilization and formulation of the above mentioned compositions.

The invention refers further to a method of preparing the presently disclosed composition, comprising producing the single compounds by chemical synthesis or isolating them from natural sources, putting the single compounds together in a suitable vessel in relative amounts as to end up with the specified amount for each compound, mixing the compounds together, and adding additional substances for stabilization and/or formulation of this composition.

The invention refers also to the use of the same composition as a food additive or medicament for preventing, curing, or improving the symptoms of mental decline.

In another embodiment, the presently disclosed composition comprises a mixture of compounds comprising at least:

a) 800 mcg (2665 IU) of Vitamin A, 500 mg of Vitamin C, 265 mg (400 IU) of Vitamin E, 50 mcg of Vitamin K,

b) 10 mg of Beta carotene, 6 mg of Lutein, 5 mg of Lycopene, 100 mcg of Zeaxanthin,

c) 7.5 mg of Vitamin B1, 7.5 mg of Vitamin B2, 15 mg of Niacin, 15 mg of Pantothenic acid, 7.5 mg of Vitamin B6, 200 mcg of Folic acid, 6.75 mcg of Vitamin B12,

d) 150 mcg of Selenium, 10 mg of Zinc, 120 mcg of Chromium, 2 mg of Copper, 4 mg of Manganese, 100 mcg of Iodine, 100 mcg of Molybdenum, and

e) 200 mcg of Biotin, 500-1000 mg of Betaine, 100 mg of Oligoproanthocyanidins (OPC), and 150 mg of Polyphenol complex.

The composition may further comprise additional substances for the purpose of stabilization and formulation of the above mentioned compositions.

The invention refers further to a method of preparing the presently disclosed composition, comprising producing the single compounds by chemical synthesis or isolating them from natural sources, putting the single compounds together in a suitable vessel in relative amounts as to end up with the specified amount for each compound, mixing the compounds together, and adding additional substances for stabilization and/or formulation of this composition.

The invention refers also to the use of this same composition as food additive or medicament for preventing, curing, or improving the symptoms of age-related eye disease.

We claim:

1. A composition comprising at least the following compounds:
a) 800 mcg of Vitamin A, 1000 mg of Vitamin C, 400 IU of Vitamin E, 200 mcg of Vitamin K,
b) 10 mg of Beta carotene, 6 mg of Lutein, 5 mg of Lycopene, 100 mcg of Zeaxanthin,
c) 7.5 mg of Vitamin B1, 7.5 mg of Vitamin B2, 15 mg of Niacin, 15 mg of Pantothenic acid, 7.5 mg of Vitamin B6, 200 mcg of Folic acid, 6.75 mcg of Vitamin B12,
d) 200 mcg of Selenium, 10 mg of Zinc, 120 mcg of Chromium, 2 mg of Copper, 4 mg of Manganese, 100 mcg of Iodine, 100 mcg of Molybdenum, and
e) 200 mcg of Biotin, 450 mg of Betaine, 100 mg of Oligoproanthocyanidins (OPC), 150 mg of Polyphenol complex, 600 to 5000 mg of mixed Omega 3 and
6, 4 to 6 g of an Oligosaccharide (FOS) , and 4 to 6 g of insulin;
wherein each compound is present in an amount not exceeding 15% (w/w) above or 15% (w/w) below the specified amount.

2. The composition of claim 1, further comprising additional substances for the purpose of stabilization and formulation of the composition.

3. The composition of claim 1, wherein the oligosaccharide (FOS) is oligo fructose or beta glucan, or a combination thereof.

4. The composition of claim 1, prepared by the process of first producing the individual compounds by chemical synthesis or isolating them from natural sources, putting the single compounds together in a suitable vessel in relative amounts as to end up with the specified amount for each compound, mixing the compounds after having put them together, and adding additional substances for stabilization and formulation of the composition.

4. The composition of claim 1, wherein the composition is provided as a food additive or in a medicament.

5. The composition of claim 4, wherein the composition is used to prevent, cure, or treat diabetes.

6. A composition comprising at least the following compounds:
a) 800 mcg of Vitamin A, 500 mg of Vitamin C, 200 IU of Vitamin E, 200 mcg of Vitamin K,
b) 10 mg of Beta carotene, 6 mg of Lutein, 5 mg of Lycopene, 100 mcg of Zeaxanthin,
c) 7.5 mg of Vitamin B1, 7.5 mg of Vitamin B2, 15 mg of Niacin, 15 mg of Pantothenic acid, 7.5 mg of Vitamin B6, 200 mcg of Folic acid, 6.75 mcg of Vitamin B12,
d) 200 mcg of Selenium, 10 mg of Zinc, 120 mcg of Chromium, 2 mg of Copper, 4 mg of Manganese, 100 mcg of Iodine, 100 mcg of Molybdenum,
e) 200 mcg of Biotin, 250 mg of Oligoproanthocyanidins (OPC), 150 mg of Polyphenol complex, 600 mg of Omega 3, 100 mg of Co-Q10, and
f) 1000 mg of Glucosamine;
wherein each compound is present in an amount not exceeding 15% (w/w) above or 15% (w/w) below the specified amount.

7. The composition of claim 6, further comprising additional substances for the purpose of stabilization and formulation of the composition.

8. The composition of claim 6, prepared by the process of producing the individual compounds by chemical synthesis or isolating them from natural sources, putting the compounds together in a suitable vessel in relative amounts as to end up with the specified amount for each compound, mixing the compounds after having put them together, and adding additional substances for stabilization and formulation of this composition.

9. The composition of claim 6, wherein the composition is provided as a food additive or a medicament.

10. The composition of claim 6, wherein the composition is used to prevent, cure, or treat osteo-arthritis.

11. A composition comprising at least the following compounds:
a) 800 mcg of Vitamin A, 500 mg of Vitamin C, 20 mcg of Vitamin D, 200 mcg of Vitamin K,
b) 10 mg of Beta carotene, 6 mg of Lutein, 5 mg of Lycopene, 100 mcg of Zeaxanthin,
c) 7.5 mg of Vitamin B1, 7.5 mg of Vitamin B2, 15 mg of Niacin, 15 mg of Pantothenic acid, 7.5 mg of Vitamin B6, 200 mcg of Folic acid, 6.75 mcg of Vitamin B12,
d) 200 mcg of Selenium, 10 mg of Zinc, 100 mg of Calcium, 50 mg of Magnesium, 120 mcg of Chromium, 2 mg of Copper, 4 mg of Manganese, 100 mcg of Iodine, 100 mcg of Molybdenum,
e) 200 mcg of Biotin, 250 mg of Oligoproanthocyanidins (OPC), 150 mg of Polyphenol complex, 40 mg of Isoflavones, 100 mg of Co-Q10, and
f) 1000 mg of Glucosamine;
wherein each compound is present in an amount not exceeding 15% (w/w) above or 15% (w/w) below the specified amount.

12. The composition of claim 11, further comprising additional substances for the purpose of stabilization and formulation of the composition.

13. The composition of claim 11, wherein the Isoflavone is genistein, daidzein, or a combination thereof.

14. The composition of claim 11, prepared by the process of producing the individual compounds by chemical synthesis or isolating them from natural sources, putting the compounds together in a suitable vessel in relative amounts as to end up with the specified amount for each compound, mixing the compounds after having put them together, and adding additional substances for stabilization and formulation of the composition.

15. The composition of claim 11, wherein the composition is provided as a food additive or a medicament.

16. The composition of claim 11, wherein the composition is used to prevent, cure, or treat osteoporosis.

17. A composition comprising at least the following compounds:
a) 800 mcg of Vitamin A, 1000 mg of Vitamin C, 15 mcg of Vitamin D, 400 IU of Vitamin E, 200 mcg of Vitamin K,
b) 10 mg of Beta carotene, 6 mg of Lutein, 5 mg of Lycopene, 100 mcg of Zeaxanthin,
c) 7.5 mg of Vitamin B1, 7.5 mg of Vitamin B2, 15 mg of Niacin, 15 mg of Pantothenic acid, 7.5 mg of Vitamin B6, 200 mcg of Folic acid, 6.75 mcg of Vitamin B12,
d) 200 mcg of Selenium, 10 mg of Zinc, 100 mg of Calcium, 50 mg of Magnesium, 120 mcg of Chromium, 2 mg of Copper, 4 mg of Manganese, 100 mcg of Iodine, 100 mcg of Molybdenum, and
e) 200 mcg of Biotin, 1000 mg of Betaine, 250 mg of Oligoproanthocyanidins (OPC), 150 mg of Polyphenol complex, 500 mg of curcuminoids, 100 mg of ginkgo biloba, 600 to 10,000 mg of mixed Omega 3, and 60-90 mg of Co-Q10;
wherein each compound is present in an amount not exceeding 15% (w/w) above or 15% (w/w) below the specified amount.

18. The composition of claim 17, further comprising additional substances for the purpose of stabilization and formulation of the composition.

19. The composition of claim 17, prepared by the process of producing the individual compounds by chemical synthesis or isolating them from natural sources, puffing the compounds together in a suitable vessel in relative amounts as to end up with the specified amount for each compound, mixing the compounds after having put them together, and adding additional substances for stabilization and formulation of the composition.

20. The composition of claim 17, wherein the composition is provided as a food additive or a medicament.

21. The composition of claim 17, wherein the composition is used to prevent, cure, or treat asthma.

22. A composition comprising at least the following compounds:
a) 800 mcg of Vitamin A, 500 mg of Vitamin C, 200 IU of Vitamin E, 200 mcg of Vitamin K,
b) 10 mg of Beta carotene, 6 mg of Lutein, 5 mg of Lycopene, 100 mcg of Zeaxanthin,
c) 7.5 mg of Vitamin B1, 7.5 mg of Vitamin B2, 15 mg of Niacin, 15 mg of Pantothenic acid, 7.5 mg of Vitamin B6, 200 mcg of Folic acid, 6.75 mcg of Vitamin B12,
d) 200 mcg of Selenium, 10 mg of Zinc, 120 mcg of Chromium, 2 mg of Copper, 4 mg of Manganese, 100 mcg of Iodine, 100 mcg of Molybdenum, and
e) 200 mcg of Biotin, 1000 mg of Betaine, 250 mg of Oligoproanthocyanidins (OPC), 150 mg of Polyphenol complex, 40 mg of Isoflavones, 600 mg of Omega 3, 4 to 6 g of Oligosaccharides (FOS), 4 to 6 g of insulin, and 60-90 mg of Co10;
wherein each compound is present in an amount not exceeding 15% (w/w) above or 15% (w/w) below the specified amount.

23. The composition of claim 22, further comprising additional substances for the purpose of stabilization and formulation of the composition.

24. The composition of claim 22, wherein the oligosaccharide (FOS) is oligo fructose or beta glucan, or a combination thereof.

25. The composition of claim 22, wherein the isoflavone is genistein, daidzein, or a combination thereof.

26. The composition of claim 22, prepared by the process of first producing the individual compounds by chemical synthesis or isolating them from natural sources, putting the single compounds together in a suitable vessel in relative amounts as to end up with the specified amount for each compound, mixing the compounds after having put them together, and adding additional substances for stabilization and formulation of the composition.

27. The composition of claim 22, wherein the composition is provided as a food additive or in a medicament.

28. The composition of claim 22, wherein the composition is used to prevent, cure, or treat mental decline.

29. A composition comprising at least the following compounds:
a) 800 mcg of Vitamin A, 500 mg of Vitamin C, 400 IU of Vitamin E, 50 mcg of Vitamin K,
b) 10 mg of Beta carotene, 6 mg of Lutein, 5 mg of Lycopene, 100 mcg of Zeaxanthin,
c) 7.5 mg of Vitamin B1, 7.5 mg of Vitamin B2, 15 mg of Niacin, 15 mg of Pantothenic acid, 7.5 mg of Vitamin B6, 200 mcg of Folic acid, 6.75 mcg of Vitamin B12,
d) 150 mcg of Selenium, 10 mg of Zinc, 120 mcg of Chromium, 2 mg of Copper, 4 mg of Manganese, 100 mcg of Iodine, 100 mcg of Molybdenum, and
e) 200 mcg of Biotin, 500-1000 mg of Betaine, 100 mg of Oligoproanthocyanidins (OPC), and 150 mg of Polyphenol complex;
wherein each compound is present in an amount not exceeding 15% (w/w) above or 15% (w/w) below the specified amount.

30. The composition of claim 29, further comprising additional substances for the purpose of stabilization and formulation of the composition.

31. The composition of claim 29, prepared by the process of producing the individual compounds by chemical synthesis or isolating them from natural sources, putting the compounds together in a suitable vessel in relative amounts as to end up with the specified amount for each compound, mixing the compounds after having put them together, and adding additional substances for stabilization and formulation of this composition.

32. The composition of claim 29, wherein the composition is provided as a food additive or a medicament.

33. The composition of claim 29, wherein the composition is used to prevent, cure, or treat age-related eye disorders.
